(19) United States
(12) Patent Application Publication
(10) Pub. No.: US 2006/0084723 A1
(43) Pub. Date: Apr. 20, 2006

(54) Nanocomposites, method of production, and method of use

(75) Inventors: Andreas Hartwig, Ritterhude (DE); Monika Sebald, Ritterhude (DE)

Correspondence Address: GREENBLUM & BERNSTEIN, P.L.C., 1950 ROLAND CLARKE PLACE, RESTON, VA, 20191, US

(73) Assignee: Fraunhofer Gesellschaft zur Foederung der Angewandten Forschung e.V., Muenchen (DE)

(21) Appl. No.: 11/071,258

(22) Filed: Mar. 4, 2005

Related U.S. Application Data
(63) Continuation of application No. PCT/DE03/02933, filed on Sep. 4, 2003.

(30) Foreign Application Priority Data
Sep. 7, 2002 (DE) 102 41 510.2

(51) Int. Cl. B32B 5/16; A61K 6/083; C08K 9/06

(52) U.S. Cl. 523/212; 428/402; 106/35; 523/115

(57) ABSTRACT

Method for production of nanocomposites from nanopowders present in agglomerated form and organic binders. Through surface modification of the nanofillers in an organic medium it is possible to divide the agglomerates permanently to such an extent that transparent nanocomposites can be preserved. The modified nanopowder is preferably isolated as a dry intermediate. The production of the disclosed nanocomposites is simpler than the production of nanocomposites by the sol-gel technique and in addition is more flexible and has wider applicability. An important application for the nanocomposites is scratch-resistant paints.

### CROSS-REFERENCE TO RELATED APPLICATIONS

This application is a continuation of International Patent Application No. PCT/DE03/02933, filed Sep. 4, 2003, the disclosure of which is expressly incorporated by reference herein in its entirety, and which published as WO 2004/024811 A2 on Mar. 25, 2004, and claims priority of German Patent Application No. 102 41 510.2, filed Sep. 7, 2002.

### BACKGROUND OF THE INVENTION

1. Field of the Invention

The invention relates to composites of nano-scale fillers and binders, methods for their production, and methods of using the composites.

2. Discussion of Background Information

According to the prior art, nanocomposites are obtained either with the aid of the so-called sol-gel method or by the mechanical incorporation of agglomerated nanofillers.

In the sol-gel method, alkoxysilanes are hydrolyzed and the silanols formed condense slowly under the cleavage of water to form particles with diameters of several nanometers. When tetraalkoxysilanes are used in this process, unfunctionalized nanoparticles of silicon dioxide are obtained hereby. The synthesis of particles takes place primarily through the Stöber process (W. Stöber, J. Coll. Interf. Sci. 26 (1968) 62). When trialkoxysilanes with a further functional group are used, the resulting nanoparticles carry the corresponding functional groups. When these groups are suitably selected, they are then capable of reacting with an organic matrix. When the co-reactants are suitably selected, it is also possible to carry out the synthesis of the nanoparticles directly in the organic matrix.

The chief disadvantages of this method for the production of nanocomposites are the high cost of raw materials, since the entire particles are produced from the expensive silane, and the difficult process control. The resulting particles have a very uniform size distribution, but this is not of importance in most applications.

On the other hand, composites of agglomerated nanoparticles can be produced in an organic matrix. The most frequently used agglomerated nanofiller is silicon dioxide produced by flame pyrolysis. However, due to the high interaction of the particles, only low degrees of filling can be achieved and the material has a great influence on the flow behavior of the modified organic matrix. The silicon dioxide produced by flame pyrolysis is therefore used customarily as a thixotroping agent.

In order to achieve an improved wetting of the surface of the agglomerated nanofillers, a surface treatment with silanes in the gas phase is sometimes described. Alternatively, solutions of the silanes in alcohols are sprayed onto the dry powders. Both techniques of surface modification lead to a less thixotroping effect of the treated nanofillers and to a beginning dispersion of the agglomerates in the organic binder. However, the measures are not sufficient to make available largely scattered nanoparticles in an organic binder.

### SUMMARY OF THE INVENTION

The present invention relates to the technical problem of surmounting the disadvantages of the prior art and of making available nanocomposites that are composed of nanoparticles in an organic matrix as well as to cost-effective methods for their production. In particular, the use of cost-intensive components in large quantities is to be dispensed with.

The present invention relates to a method for production of nanocomposites, comprising organically modifying agglomerated nanofillers in an organic solvent with at least one of a silane, chlorosilane, silazane, titanate and zirconate to form organically modified nanofillers, the agglomerated nanofillers comprising oxidic or nitridic compounds produced by flame pyrolysis or by precipitation, and no acid is added in the organic modification of the agglomerated nanofillers, and incorporating the organically modified nanofillers into an organic binder.

The at least one of a silane, chlorosilane, silazane, titanate, and zirconate can have the general formulas Si(OR′)ₙR₄₋ₙ, SiClₙRₙ₋₄, (RₘR″ₘ₋₃Si)₂NH, Ti(OR′)ₙR₄₋ₙ, and Zr(OR′)ₙR₄₋ₙ, where m=1, 2, or 3 and n=1, 2, or 3, and R, R′ and R″ are any organic functional group, and n can preferably be 3.

The at least one of a silane, chlorosilane, silazane, titanate, and zirconate can be a trialkoxysilane, and the trialkoxysilane can comprise at least one of trimethoxy-, triethoxy-, and triisopropoxysilane.

The group R can enter into a chemical reaction with the binder or can have a high affinity to the binder.

R can be an acrylate or methacrylate group and the binder can be acrylate- or methacrylate-based.

R can have an epoxide-, amino-, carboxylic acid-, thiol-, or alcohol group and the binder can be an epoxide-based binder.

R can contain a polymerizable double bond and the binder can contain styrene or an unsaturated polyester.

R can contain an amino-, alcohol-, thiol-, isocyanate-, or carboxylic acid group and the binder can contain isocyanate groups.

R can be a hydrophobic grouping, and the binder can contain silicone, and the hydrophobic grouping can comprise trimethylsilyl.

The organic modification can be carried out directly in the binder for production of the nanocomposite as a solvent.

Additional mechanical energy can be introduced at least one of during the modification of the nanofillers and during the incorporation of the modified nanofillers into the binder. The additional mechanical energy can be applied by ultrasound, a high-speed stirrer, a dissolver, a bead mill, or a rotor-stator mixer.

The modified nanofillers can be incorporated into the binder in a form of a dispersion in the organic solvent.

The modified nanofillers can be incorporated into the binder in a form of a dry powder.

The nanofillers can be incorporated into monomers used for production of thermoplastics as the binder, and polymerizing the monomers.

The polymerization of the binder containing nanofillers can take place in an aqueous dispersion or emulsion.

The binder can comprise a thermoplastic and the nanofillers can be incorporated into a melt of the thermoplastic.

Organically modified nanofillers of varied identity or particle size distribution can be combined with one another.

The organically modified nanofillers can be combined with lamellar or acicular nanofillers.

The present invention also relates to paint, adhesive, sealing compound, coating, or plastic molded part comprising the nanocomposite according to the present invention.

The paint, adhesive, sealing compound, coating, or plastic molded part can be for aircraft construction, in electronics, for automotive finishes, for varnishing transparent plastics, or as parquet floor varnish.

The present invention also relates to a secondary dispersion comprising the nanocomposite according to the present invention.

The present invention also relates to a dental material comprising the nanocomposite according to the present invention.

The present invention also relates to a nanocomposite produced by the method according to the present invention.

### BRIEF DESCRIPTION OF THE DRAWINGS

FIG. 1 shows the testing of the obtained polymer in the transmission electron microscope; and

FIG. 2 shows a TEM micrograph of the nanocomposite cured through UV radiation

### DETAILED DESCRIPTION OF THE INVENTION

To surmount the prior art, commercially available agglomerated nanopowders are dispersed in an organic solvent and modified organically at the surface with a silane, chlorosilane, silazane, titanate, and/or zirconate for the production of the nanocomposites of the invention. In the further steps, the dispersion of the modified nanoparticles in the solvent is used directly, or preferably the solvent is drawn off and then the dry nanopowder is incorporated into the organic binder. This method causes the agglomerates to be permanently reduced to such an extent that transparent nanocomposites can be produced.

Nanocomposites are understood herein to mean mixtures of a binder or a polymer matrix and organically modified nanofillers. The agglomerates customarily formed from nanofillers are thereby surprisingly divided until at least 60%, preferably at least 80%, of the agglomerates have a particle diameter of less than 300 nm. In most cases it is even possible to achieve individual particles and agglomerates with diameters of less than 100 nm.

Compared with conventionally produced composites of binders and fillers, the systems according to the invention have the advantages associated with nanofillers. These are the possibility of making available transparent but nonetheless filled composites and an improvement in the mechanical and thermal properties. On the other hand, the composites according to the invention are superior to the so-called sol-gel materials due to a simplified production, more universal applicability, and the possibility of making dry nanofillers available. Compared with the use of strongly agglomerated nanoparticles as fillers, as are present if no modification or gas phase modification of the surface has taken place, the nanoparticles according to the invention organically modified in an organic solvent have the advantage that, compared with the unfilled binder, they have only a slight influence on the rheological properties of the nanocomposites produced with them. In contrast, the nanofillers according to the prior art have an effect that is usually strongly thixotroping and thickening.

Furthermore, the nanocomposites according to the invention have the advantage of cost-effective production. The fillers needed for the production are produced from available agglomerated nanoparticles by organic surface treatment. Compared with the sol-gel method known from the prior art, this method has the advantage that it is possible to use distinctly smaller amounts of the expensive organic components, since they are needed only for the surface treatment and not for the production of the entire particles.

The agglomerated nanopowders to be used as starting material are in particular oxidic or nitridic compounds produced by flame pyrolysis or by precipitation. However, differently based agglomerated nanofillers such as, e.g., barium sulfate or barium titanate are also suitable. It is preferred to use oxides and particularly preferred to use silicon dioxide produced by flame pyrolysis.

The organic modification of the surface in the solvent takes place by treating with a silane, chlorosilane, silazane, titanate, or zirconate. These preferably have the general formulas Si(OR′)ₙR₄₋ₙ, SiClₙRₙ₋₄, (RₘR″ₘ₋₃Si)₂NH, Ti(OR′)ₙR₄₋ₙ, and Zr(OR′)ₙR₄₋ₙ, where m and n are 1, 2, or 3, preferably n=3. The group R′ bound via the oxygen, like R″, is any organic functional group, preferably an alkyl group and particularly preferred methyl, ethyl, or isopropyl. These groups are cleaved in the form of the alcohol during the organic modification. In the case of modification with the silazane, ammonia is cleaved, and in the case of the chlorosilanes, hydrochloric acid. The alcohol formed, the hydrochloric acid, or the ammonia is no longer contained in the nanocomposite produced in the subsequent steps.

The functional group R is preferably any organic group and is bound directly via a carbon atom to the silicon, titanium, or zirconium. When n or m are 1 or 2, the groups R can be the same or different. R is selected such that the group can react chemically with the monomer used to produce the nanocomposite or has a high affinity to the organic binder.

For the production of nanocomposites based on acrylates or methacrylates, R preferably contains an acrylate or methacrylate group and is particularly preferred —(CH₂)₃—S—(CH₂)₂—C(═O)O—(CH₂)ₙ—OC(═O)—CH═CH₂ where n=1 to 12 and —CH₂)₃—OC(═O)—C(CH₃)═CH₂.

For the production of nanocomposites based on epoxides, R preferably contains an epoxide group or an amino-, carboxylic acid-, thiol-, or alcohol group that can react with an epoxide group. R is particularly preferred to be 2-(3,4-epoxycyclohexyl)ethyl, 3-glycidoxypropyl, 3-aminopropyl, and 3-mercaptopropyl.

In the production of nanocomposites based on unsaturated polyesters or styrene-containing resins, R preferably contains a reactive double bond. In this application, R is particularly preferred to be vinyl or styryl or contains a vinyl or styryl group.

For the production of nanocomposites based on urethanes, polyureas or other polymer systems based on isocyanates, R preferably contains an isocyanate-, amino-, alcohol-, thiol-, or carboxylic acid group. In this case R is particularly preferred to be 3-isocyanatopropyl, 3-aminopropyl, and 3-mercaptopropyl.

The mixture of organically modified nanofillers and an organic binder is hardened by the methods customary for the respective binder. This is typically a thermal reaction at room temperature or elevated temperature, a reaction with atmospheric moisture, or UV- or electron beam curing.

During the production of the nanocomposites, the organically modified nanofillers according to the invention can be used alone or as a combination of nanofillers of different substances or different particle size distribution. In order to be able to achieve particularly high filler contents, it is advisable to combine nanofillers of different particle size distribution and optionally even to add microfillers. Moreover the nanocomposites according to the invention can contain additives customary for polymer materials, such as antioxidants, flow-control agents, dispersing agents, dyes, pigments, other fillers, or stabilizers.

The solvent in which the modification of the nanofillers is carried out is preferably a polar aprotic solvent and particularly preferred is acetone, butanone, ethyl acetate, methyl isobutyl ketone, tetrahydrofuran, and diisopropyl ether.

Furthermore, the direct modification in the organic binders to be used for the production of the nanocomposites is a particularly preferred method. In this case the monomers to be polymerized as individual components or as a formulation are the solvent to be used.

To accelerate the organic modification of the nanofillers in the organic solvent, an acid, e.g. hydrochloric acid, can be added as a catalyst. However, it has surprisingly proved that the quality of the nanocomposites produced is better when no acid is added. In each case catalytic amounts of water, preferably between 0.1% and 5%, must be present in order to carry out the modification. This water is frequently already present as an adsorbate at the surfaces of the agglomerated nanofillers used as starting material. To assist the reaction, further water can be added, e.g. also in the form of a dilute acid.

An advantageous development of the invention is the modification of the surface of the nanofillers with dyes. In this case the group R of the siloxane, silazane, titanate, or zirconate used for the modification is a dye or can react with a dye. The binding of the dye to the surface of the nanofiller can take place both via a covalent bond and via an ionic bond. Surprisingly, it has proved that the plastic components and paints that contain the nanofillers modified with dyes, have a better fading resistance than the plastic components and paints that contain the same dyes without binding to the nanofillers. In this manner it is possible to make available transparent polymer materials that are dyed so as to be fade-resistant.

The method according to the invention is also particularly suited to make available the filler particles that can be excited by fields for the production of thermoset plastics in accordance with DE 102 10 661 A1. In particular, nanocomposites are obtainable in which the agglomerated nanofillers can be excited by electrical, magnetic, and/or electromagnetic fields. Adhesive compositions according to DE 102 10 661 A1 are curable under mild conditions to produce a resistant adhesive bond with high strength and can be dissolved again without the long-term resistance of the adhesive bond having to suffer therefrom. Due to the organic modification according to the invention in a solvent, the excitable nanoparticles can be distributed particularly homogeneously in such adhesive compositions.

In order to accelerate the breakdown of the agglomerates during the organic modification in the organic solvent, an additional application of mechanical energy can be carried out with the customary methods before or during the modification. This can take place, e.g., through ultrasound, a high-speed stirrer, a dissolver, a bead mill, or a rotor-stator mixer. This is the preferred method when higher-viscosity solvents are used, particularly when the organic binder for the production of the nanocomposites is used directly as a solvent. If the binder is not used as a solvent, the binder to be used can be poured directly with the dispersion of the organically modified nanofiller in the organic solvent. In this case the solvent is drawn off after the production of the mixture of binder and organically modified nanofiller, or not until the later use of the nanocomposite composed of binder and nanofiller. The latter is a viable method, particularly with solvent-containing paints based on the nanocomposites according to the invention. However, the organically modified nanofiller is preferably freed of the solvent and is further processed as a dry powder. In this case the dry organically modified nanofiller powder is then added to the binder and incorporated under application of mechanical energy. The incorporation can be carried out, e.g., by ultrasound, a high-speed stirrer, a dissolver, a bead mill, a roller mill, or a rotor-stator mixer.

In the production of nanocomposites with thermoplastics as binders, the organically modified nanofiller is preferably incorporated into the monomers on which the thermoplastic is based. Then these monomers are polymerized conventionally, whereby the nanocomposites according to the invention result. For example, the organically modified nanofiller is incorporated into methyl methacrylate. In the subsequent polymerization, a filled poly(methyl methacrylate) results. In contrast to conventionally filled poly(methyl methacrylate), however, this is transparent and compared with the unfilled material has improved mechanical properties (for example scratch resistance, tensile strength, and bending strength). A nanocomposite based on polystyrene as a binder is named as a further example. In this case the organically modified nanofiller is incorporated into styrene and then polymerized conventionally. If a siloxane, chlorosilane, silazane, titanate, or zirconate in which the group R can polymerize together with the monomer is used in the modification of the nanofillers, the nanocomposite formed is crosslinked. In this case the organically modified nanoparticles act as crosslinker particles. If the groups R cannot react with the monomer, the nanocomposite formed is thermoplastic.

However, the modified nanoparticles can also be readily incorporated into the melt of thermoplastics. This takes place particularly effectively with an extruder or twin-screw extruder. Thus a polystyrene melt can be effectively modified during the extrusion by incorporating pyrogenic silica treated with phenyltriethoxysilane in butanone.

Polymer dispersions are needed for many applications. It has not been possible hitherto to modify these with nanoparticles. Polymer dispersions modified with nanofillers can be produced according to the invention. This is accomplished by incorporation of the surface-modified nanofillers of the invention into the monomer on which the polymer dispersions are based, subsequent dispersion of this monomer/nanofiller mixture in water with the addition of a surfactant, and subsequent thereto, dispersion polymerization or emulsion polymerization. The surface modification of the nanofiller preferably takes place thereby directly in the monomer or monomer mixture. When a silane that contains a group that can be incorporated during polymerization is used for the surface modification, the nanofiller particles can be bound chemically to the polymer formed. Of course, any desired gradations between silanes with reactive and nonreactive groups can be undertaken here. For example, polystyrene latex modified with nanofiller particles or poly(styrene-co-butadiene) latex can be produced with the described method by incorporating pyrogenic silica into the monomer under simultaneous surface treatment with phenyltriethoxysilane, dispersing the filled monomer in water under addition of a surfactant, and subsequent thermal polymerization with the aid of a radical initiator. With secondary dispersions, in an analogous manner the nanofiller according to the invention is incorporated into the polymer on which the dispersion is based and then the dispersion is produced as with the nonmodified polymer.

Surprisingly, it has proved that the properties of the nanocomposites with the organically modified nanofillers can be even further improved if additionally lamellar or acicular nanofillers are added, preferably in amounts of between 0.1 and 10%. Boehmite, bentonite, montmorillonite, vermiculite, hectorite, and laponite are preferably used for this. In order to obtain a good compatibility of the lamellar nanofillers with the organic binder, the lamellar nanofillers are organically modified according to the prior art. The addition of the lamellar or acicular nanofillers to the nanocomposites of the invention leads to a further increase in the mechanical strength. When the nanocomposites are used as adhesive or sealing compound, the further increase in heat conductivity, improvement in mechanical strength, and reduction in combustibility through the addition of the lamellar nanofillers are to be emphasized as a further improvement of properties.

The nanocomposites according to the invention can be used particularly advantageously in the form of adhesives, sealing compounds, paints, coatings, and plastic molded parts.

When used as paint, the particular advantage of the nanocomposites according to the invention, compared with the unfilled paints, is the improved scratch resistance and abrasion resistance. At the same time, the transparency is preserved. This combination of properties is in demand particularly in the use of finishing paints, e.g. for automotive finishes and parquet floor varnishes. Another application case is the varnishing of transparent plastics, in particular poly(methyl methacrylate), polycarbonate, and polystyrene, in order to improve the scratch resistance of the surface without impairing the transparency.

When the nanocomposites according to the invention are used as a scratch-resistant varnish, a filler content of between 1 and 80% by wt, preferably between 5 and 50% by wt, and particularly preferred between 20 and 50% by wt, is suitable. Such varnishes are particularly suitable for endowing automobile windows made of plastic, preferably of polycarbonate, with a scratch-resistant finish. A further preferred use of the nanocomposites of the invention is parquet floor varnishes. The hardening of the varnishes is preferably induced thermally by polyaddition, by oxidative drying, or by UV-induced polymerization. For endowing plastic parts with scratch resistance, however, the entire component can also be composed of the nanocomposites of the invention or can be built up in the form of layers of unfilled plastic and the nanocomposite.

In the case of hydrophobic soft coatings, e.g., silicone coatings on a great variety of substrates (e.g., backing papers, foils, plastic components), both the surface adhesion (e.g., for contaminants or pressure-sensitive adhesives) and the mechanical properties (e.g., abrasion, resistance) can be modified through the modified nanoparticles of the invention. This also has in particular an influence on the haptics of the polymer and thus can preferably be used on handles and other objects with which hands come into contact. In order to bind the nanoparticles into the polymer network, it is advisable that they carry both purely hydrophobic groups (in particular —Si(CH₃)₃) and reactive groups (e.g., vinyl in the case of silicones crosslinking on double bonds through hydrosilane addition).

When the nanocomposites according to the invention are used as sealing compounds and adhesives, the improvement in the mechanical strength and in the heat conductivity is of particular significance. Special types of sealing compounds or adhesives are needed in the field of dentistry. Polymer materials that are particularly abrasion-resistant and can be subjected to high mechanical stresses are needed in the filling and veneering of teeth, as well as, for example, in the fabrication of prostheses. These materials can be made available with the nanocomposites of the invention. The reactive materials known according to the prior art are the preferred basis of such materials. The methacrylates and acrylates are to be named in particular. The curing is preferably carried out photochemically, to which end suitable photoinitiators (e.g. camphorquinone) are added.

The nanocomposites according to the invention can furthermore be used advantageously in aircraft construction, in electronics, for automotive finishes, and for varnishing transparent plastics (e.g. automobile windows made of polycarbonate).

Dispersions modified with nanofillers are preferably used for water-based paints, coatings, and adhesives—in particular contact adhesives and pressure-sensitive adhesives. Solvent-based polymer preparations are frequently also needed for the same applications. These can be made available either by incorporating the modified nanofillers or by modifying the nanofillers in the finished polymer solution. On the other hand, the modification can also be carried out in the monomer or the monomer/solvent mixture and polymerization can take place only subsequently.

### EXAMPLES

Without restricting the generality, the invention is explained in more detail below based on several examples.

### Example 1

Production of a nanocomposite from an epoxy resin with a modified nanofiller:

a) Organic Modification of the Agglomerated Nanofiller

40.3 g of Aerosil 200 was suspended in butanone (650 g) for 5 min and 25.5 g of 2-(3,4-epoxycyclohexyl)ethyltrimethoxysilane (ECHTMO) and 5.6 g of 1 N hydrochloric acid were added dropwise to the catalysis. The mixture was stirred for 48 h. Then the butanone was drawn off completely on the rotary evaporator. A loose porous white powder was obtained.

b) Production of a Masterbatch in Epoxy Resin

A masterbatch with 50% by wt of the modified filler in the epoxy resin ERL 4221 (Union Carbide) is produced. 30.2 g of the filler modified according to a) and 1.5 g of Disperbyk-111 were added to 30 g of the epoxy resin in several portions under stirring with the Dispermat CA 40 C at 1-2 m/s. Dispersion was carried out at 8 m/s between the additions. In all, the batch was dispersed for 8.5 h at a circumferential speed of 8 m/s (125 mL vessel, 30 mm Ø dissolver disk). Then the sample was degassed on the Vacuum Dispermat at 2300 rpm for 2 h. A transparent resin system results that if necessary is diluted with further resin to the desired filler concentration.

c) Thermal Curing of the Epoxy Resin

5 g of the masterbatch produced according to b) is diluted with 5 g of the epoxy resin, and 0.1 g respectively of Rhodorsil 2074 (Rhodia) and ascorbic acid 6-hexadecanate as a thermal initiator system are dissolved. Then the sample is poured out into an aluminum dish and cured at 90° C. for 60 min and then at 120° C. for 30 min. A transparent polymer results.

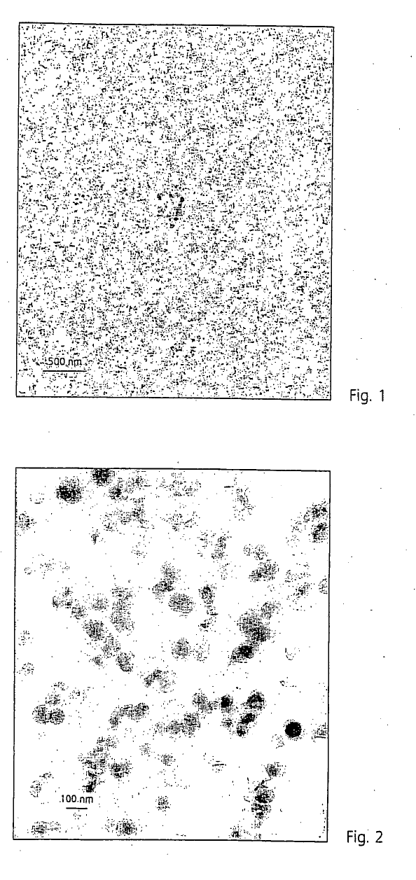

FIG. 1 shows the testing of the obtained polymer in the transmission electron microscope. The agglomerates typical for the unmodified filler are largely dispersed by the modification, which is the cause of the good transparency of the sample.

d) Photochemical Curing of the Epoxy Resin

The produced masterbatch is diluted with further epoxy resin to a filler content of 25% by wt, and 1% of the photoinitiator Sarcat CD 1010 (Sartomer) is added. The mixture cures by radiation with UV light and is used in Example 7 to test the abrasion resistance.

### Example 2

Modification of a Silicon Dioxide Produced by Flame Pyrolysis without Acid Catalysis:

Catalysis with HCl was omitted in this test. 40 g of Aerosil 200 was suspended in 600 g of butanone, the silane ECHTMO (25.2 g) was added dropwise slowly via a dropping funnel, and the mixture was stirred for 16 h. Then the butanone was drawn off completely on the rotary evaporator. The filler resulted as porous clumps that could readily be reduced with a mortar.

### Example 3

Production of a Nanofiller with Acrylate Groups:

Ethanol KOH (1.62 g KOH in 30 mL ethanol) was slowly added dropwise to 5.16 g of (3-mercaptopropyl)trimethoxysilane and 6.78 g of hexanediol diacrylate in 250 mL of ethyl acetate at 0° C. under N₂ atmosphere, so that the reaction temperature of 20° C. was not exceeded. The reaction is stopped after 5 min. An iodine test was used to test for complete conversion. The reaction solution was shaken out three times with saturated NaCl solution, after which processing the organic phase was neutral and cloudy. The Aerosil 200 was suspended in the organic phase and the reaction was catalyzed with 1 mL of 0.5 N HCl. Stirring was carried out for 24 h at room temperature and then the ethyl acetate was drawn off on the rotary evaporator. A loose white powder resulted.

### Example 4

Preparation of a Nanofiller Based on Titanium Dioxide:

25.0 g of titanium dioxide P25 (Degussa) was silanized with 3.94 g of ECHTMO. To this end the P25 was suspended in 400 g of butanone, the ECHTMO and 8.86 g of 1 N HCl were added dropwise, and the mixture was stirred on the magnetic stirrer for 24 h. Then the butanone was drawn off completely on the rotary evaporator. The modified titanium dioxide P25 resulted as a loose white powder.

### Example 5

Production of a Nanocomposite Based on an Epoxide and a Silicon Dioxide with Quite Large Primary Particles Produced by Flame Pyrolysis:

a) Modification of the Nanofiller

90.9 g of Aerosil OX 50 was suspended in 450 g of butanone for 5 min, 14.35 g of ECHTMO and 3.1 g of 1 N HCl were added dropwise, and the mixture was stirred for 48 h. Then the butanone was drawn off completely on the rotary evaporator. A loose porous white powder was obtained.

b) Incorporation into the Epoxy Resin

With the nanofiller produced according to Sa), a 25% by wt filled resin of ERL 4221 with 3% Disperbyk-111 (BYK Chemie) relative to the filler was produced. To this end two-thirds of the resin was introduced beforehand and the filler was added in portions; the Disperbyk-111 was added dropwise after half of the filler had been added. The individual portions of filler were added to the Dispermat CA 40 C at 1 m/s, and dispersing was carried out at 11 m/s between the additions. After 90 min the remaining third of the resin was added. Then the batch was dispersed for 7 h at a circumferential speed of 8 m/s (125 mL vessel, 30 mm Ø dissolver disk). A medium-viscosity transparent resin resulted. 1% of the photoinitiator Sarcat CD 1010 (Sartomer) is stirred in and the reactive mixture is used further in Example 7 to determine the scratch resistance.

FIG. 2 shows a TEM micrograph of the nanocomposite cured through UV radiation. The filler is present in the form of largely isolated particles.

### Example 6

Use of the Binder as an Organic Solvent:

19.5 g of Aerosil 200 and 10.8 g of methacryloxypropyltrimethoxysilane were stirred in portions into 91 g of base resin (60% of Genomer 4302, 37% of Genomer 1223, 1% of additive 99-622, 2% of photoinitiator blend Genocure LTM, all Rahn AG). The individual portions of filler were added to the Dispermat CA 40 C at 100 rpm; between the additions, dispersing was carried out for 1-2 min at a circumferential speed of 7 m/s. In this manner two-thirds of the Aerosil was dispersed in the resin. Further stirring took place overnight at 1500 rpm. Then the remaining part of the Aerosil was moistened with butanone and added. The butanone was drawn off under vacuum at 1500 rpm. The resulting resin is transparent and at 50° C. can be applied with a doctor onto a sample carrier.

### Example 7

Abrasion Resistance of Nanocomposites:

Using a Teledyne Model 5150 Taber abrasion tester, the abrasion was determined at 1000 rpm with abrasive rollers CS 17 under a total load of 2500 g for the following samples:

a) Resin from example 1d: Aerosil 200 ECHTMO 25% by wt in ERL 4221

b) Resin from example 5: Aerosil OX 50 ECHTMO 25% by wt in ERL 4212

c) Control sample: ERL 4221 without filler

d) Resin from example 6: Aerosil 200 MEMO 25% by wt in acrylic resin

e) Control sample: acrylic resin without filler.

Production of Samples:

The samples a), b), and c) based on the epoxy resin ERL 4221 were applied with a wire-wound doctor of 60 μm onto 10×10 cm polycarbonate sheets and were cured in the BK 200 UV-radiation unit (arccure technologies) in two passes (surrounding atmosphere, 100% illumination, 28% transport speed). The acrylic samples d) and e) were heated to about 50° C., applied with a doctor with a thickness of 60 μm onto pre-heated 10×10 cm aluminum sheets, and were cured in the BK 200 UV-radiation unit (arccure technologies) in two passes (surrounding atmosphere, 100% illumination, 28% transport speed). The following abrasions were measured:

| | Abrasion |
|---|---|
| Sample | (mg) |
| c) Epoxy unfilled, comparative | 38.78 |
| a) Epoxy from example 1d | 9.12 |
| b) Epoxy from example 5 | 15.93 |
| e) Acrylic unfilled, comparative | 15.22 |
| d) Acrylic from example 6 | 4.67 |

In these examples, the abrasion of the modified coatings is less than for the unfilled base resins by a factor of 2 to 4.

### Example 8

### Comparative Example

Modification of an Acrylic with Unmodified Silicon Dioxide Produced by Flame Pyrolysis:

A base resin made of 120 parts by wt of Genomer 4302, 74 parts by wt of Genomer 1223, and 2 parts by wt of additive 99-622 (all by Rahn) is prepared. 2.8 g of Aerosil 200 (Degussa) is gradually stirred into 61.9 g of base resin with 1 mL of Disperbyk-111 (BYK) with a Dispermat. Then dispersing is continued for 3 h at 8 m/s. Even at the low filler content of 4.3% by wt, a nontransparent highly viscous thixotropic resin resulted.

### Example 9

### Comparative Example

Modification of an Epoxy Resin with a Gas-Phase-Modified Silicon Dioxide Produced by Flame Pyrolysis:

20 g of Aerosil 200 is placed in a bottle with 12.7 g of ECHTMO and thoroughly mixed on a shaker for one hour. The reaction is allowed to continue overnight and the remaining silane and the methanol formed is removed under vacuum. The reaction product is incorporated into 100 g of ERL 4221 by dispersion at a circumferential speed of 8 m/s. A highly viscous white resin system is formed.

What is claimed:

1. Method for production of nanocomposites, comprising organically modifying agglomerated nanofillers in an organic solvent with at least one of a silane, chlorosilane, silazane, titanate and zirconate to form organically modified nanofillers, the agglomerated nanofillers comprising oxidic or nitridic compounds produced by flame pyrolysis or by precipitation, and no acid is added in the organic modification of the agglomerated nanofillers, and incorporating the organically modified nanofillers into an organic binder.

2. The method for production of nanocomposites according to claim 1, wherein the at least one of a silane, chlorosilane, silazane, titanate, and zirconate have the general formulas Si(OR′)ₙR₄₋ₙ, SiClₙRₙ₋₄, (RₘR″ₘ₋₃Si)₂NH, Ti(OR′)ₙR₄₋ₙ, and Zr(OR′)ₙR₄₋ₙ, where m=1, 2, or 3 and n=1, 2, or 3, and R, R′ and R″ are any organic functional group.

3. The method for production of nanocomposites according to claim 2, wherein n is 3.

4. The method for the production of nanocomposites according to claim 2, wherein the at least one of a silane, chlorosilane, silazane, titanate, and zirconate is a trialkoxysilane

5. The method for the production of nanocomposites according to claim 4, wherein the trialkoxysilane comprises at least one of trimethoxy-, triethoxy-, and triisopropoxysilane.

6. The method for production of nanocomposites according to claim 2, wherein the group R can enter into a chemical reaction with the binder or has a high affinity to the binder.

7. The method for production of nanocomposites according to claim 2, wherein R is an acrylate or methacrylate group and the binder is acrylate- or methacrylate-based.

8. The method for production of nanocomposites according to claim 2, wherein R has an epoxide-, amino-, carboxylic acid-, thiol-, or alcohol group and the binder is an epoxide-based binder.

9. The method for production of nanocomposites according to claim 2, wherein R contains a polymerizable double bond and the binder contains styrene or an unsaturated polyester.

10. The method for production of nanocomposites according to claim 2, wherein R contains an amino-, alcohol-, thiol-, isocyanate-, or carboxylic acid group and the binder contains isocyanate groups.

11. The method for production of nanocomposites according to claim 2, wherein R is a hydrophobic grouping, and the binder contains silicone.

12. The method for production of nanocomposites according to claim 11, wherein the hydrophobic grouping comprises trimethylsilyl.

13. The method for production of nanocomposites according to claim 1, wherein the organic modification is carried out directly in the binder for production of the nanocomposite as a solvent.

14. The method for production of nanocomposites according to claim 1, wherein additional mechanical energy is introduced at least one of during the modification of the nanofillers and during the incorporation of the modified nanofillers into the binder.

15. The method for production of nanocomposites according to claim 14, wherein the additional mechanical energy is applied by ultrasound, a high-speed stirrer, a dissolver, a bead mill, or a rotor-stator mixer.

16. The method for production of nanocomposites according to claim 1, wherein the modified nanofillers are incorporated into the binder in a form of a dispersion in the organic solvent.

17. The method for production of nanocomposites according to claim 1, wherein the modified nanofillers are incorporated into the binder in a form of a dry powder.

18. The method for production of nanocomposites according to claim 1, wherein the nanofillers are incorporated into monomers used for production of thermoplastics as the binder, and polymerizing the monomers.

19. The method for production of nanocomposites according to claim 18, wherein polymerization of the binder containing nanofillers takes place in an aqueous dispersion or emulsion.

20. The method for production of nanocomposites according to claim 1, wherein the binder comprises a thermoplastic and the nanofillers are incorporated into a melt of the thermoplastic.

21. The method for production of nanocomposites according to claim 1, wherein organically modified nanofillers of varied identity or particle size distribution are combined with one another.

22. The method for production of nanocomposites according to claim 21, wherein the organically modified nanofillers are combined with lamellar or acicular nanofillers.

23. The method for production of nanocomposites according to claim 1, wherein the organically modified nanofillers are combined with lamellar or acicular nanofillers.

24. A paint, adhesive, sealing compound, coating, or plastic molded part comprising the nanocomposite according to claim 1.

25. The paint, adhesive, sealing compound, coating, or plastic molded part according to claim 24 for aircraft construction, in electronics, for automotive finishes, for varnishing transparent plastics, or as parquet floor varnish.

26. A secondary dispersion comprising the nanocomposite according to claim 1.

27. A dental material comprising the nanocomposite according to claim 1.

28. A nanocomposite produced by the method according to claim 1.
